SEQUENCE LISTING (2 sequences)

SEQ ID NO: 1 - DNA, 1881 bp, synthetic construct
  misc_feature 1..1881: note = recombinant nucleic acid molecule
  misc_feature 1..1881: note = CDS comprising a phagolysosomal domain (211-1722nt) and a stopcodon (1879-1881)
  source 1..1881: mol_type = other DNA
atgacagacgtgagccgaaagattcgagcttggggacgccgattgatgatcggcacggcagcggctgtagtccttccgggcctggtggggcttgccggcggagcggcaaccgcgggcgcgttctcccggccggggctgccggtcgagtacctgcagtctgcaaagcaatccgctgcaaataaattgcactcagcaggacaaagcacgaaagatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatcgttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgccaggtaacgcgagaaatattaatgtttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaatatgcattagcctatggaagtcagggtgatcttaatccattaattaatgaaatcagcaaaatcatttcagctgcagttctttcctctttaacatcgaagctacctgcagagttcgttaggcgcggatccggaattcgaagcttatcgatgtcgacgtag

SEQ ID NO: 2 - protein, 626 aa, synthetic construct
  REGION 1..626: note = CORRESPONDING AMINO ACID SEQUENCE OF SEQ ID NO: 1.
MTDVSRKIRAWGRRLMIGTAAAVVLPGLVGLAGGAATAGAFSRPGLPVEYLQSAKQSAANKLHSAGQSTKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIEYALAYGSQGDLNPLINEISKIISAAVLSSLTSKLPAEFVRRGSGIRSLSMST

(19) United States
(12) Patent Application Publication
(10) Pub. No.: US 2024/0277825 A1
(43) Pub. Date: Aug. 22, 2024

(54) Recombinant Mycobacterium as an Immunotherapeutic Agent for the Second-Line Therapy of Bladder Carcinoma

(71) Applicants: VAKZINE PROJEKT MANAGEMENT GMBH, Hannover (DE); SERUM INSTITUTE OF INDIA LTD., Hadapsar Maharashtra (IN)

(72) Inventor: Leander GRODE, Hannover (DE)

(73) Assignees: SERUM LIFE SCIENCE EUROPE GMBH, Hannover (DE); SERUM INSTITUTE OF INDIA LTD., Hadapsar Maharashtra (IN)

(21) Appl. No.: 18/291,147

(22) PCT Filed: Jul. 20, 2022
(86) PCT No.: PCT/EP2022/070373

§ 371 Date: Jan. 22, 2024

Related U.S. Application Data
(63) Continuation-in-part of application No. 17/667,784, filed on Feb. 9, 2022.
(60) Provisional application No. 63/224,575, filed on Jul. 22, 2021.

(30) Foreign Application Priority Data
Jul. 22, 2021 (EP) 21187253.6

(51) Int. Cl. A61K 39/04; A61K 39/00; A61P 35/00

(52) U.S. Cl. CPC A61K 39/04; A61P 35/00; A61K 2039/522; A61K 2039/523; A61K 2039/585; A61K 2039/80

(57) ABSTRACT

The invention relates to a recombinant Mycobacterium cell for use as an immunotherapeutic agent in the treatment of bladder carcinoma, particularly in the second-line treatment of non-muscle-invasive bladder carcinoma.

The invention relates to a recombinant Mycobacterium cell for use as an immunotherapeutic agent in the therapy of bladder carcinoma, particularly in the second-line therapy of non-muscle-invasive bladder carcinoma.

Urothelial bladder carcinoma is the 5th most common cancer. In the United States, about 75.000 new cases are diagnosed each year 4.5% of all new cancers, and approximately 15.600 deaths are expected. In Germany, about 16.000 new cases are diagnosed each year. Because a recurrence of disease 15 is likely in bladder carcinoma, patients must undergo surveillance for an extended period.

Most bladder carcinomas begin in transitional epithelial cells that make up the inner lining of the bladder. As these tumors grow, they can invade the surrounding connective tissue and muscle. In advanced disease, tumors spread beyond the bladder to nearby lymph nodes or pelvic organs or metastasize to more distant organs such as lung, liver, and bone.

The overall 5-year survival rate for bladder carcinoma is 77%, and this rate has not changed significantly over the last 10 years. When considered by stage, the 5-year relative survival rates for patients with tumors restricted to the inner layer of the bladder are 96% and 69%, respectively. The rates drop to 34% for those with disease that has spread locally beyond the bladder and to 6% with distant metastases.

In the treatment of bladder carcinoma, tumor recurrence is a major concern, even for patients with low-grade disease and requires extensive follow-up. Better treatments, such as novel immunotherapies, might reduce recurrence rates and improve the survival of patients with bladder carcinoma.

For patients with non-muscle invasive bladder carcinoma, treatment usually involves a surgical removal of the tumor followed by chemotherapy, usually mitomycin C, within the bladder (so-called intravesical chemotherapy). After recovering from surgery, patients with a lower risk of disease progression may undergo surveillance or additional intravesical chemotherapy. Patients with moderate-to-high-grade disease often receive intravesical immunotherapy with an attenuated live bacterium Bacillus Calmette Guérin (BCG). This therapy is designated “conventional” or “standard” BCG therapy in the following. Conventional BCG therapy was the first FDA-approved immunotherapy and helps reduce the risk of bladder carcinoma recurrence by stimulating an immune response that targets the bacteria as well as any bladder carcinoma cells. Despite the proven efficacy of conventional BCG treatment, about 35 to 45% of patients experience disease recurrence by 5 years and in about 10 to 13% of patients the disease progresses of (Oddens et al., Eur Urol 63 (2013, 462-472).

Recurrence and progression to muscle-invasive disease may lead to additional chemotherapeutical, surgical, and radio-oncological interventions including cisplatin-based chemotherapy, transurethral resection of the bladder (TURB), cystectomy and chemo-radiotherapy. Recurrent bladder carcinoma may be treated with combination therapy regimens, including gemcitabine plus cisplatin or methotrexate, vinblastine, doxorubicin plus cisplatin.

An alternative option for recurrent patients is a re-treatment with conventional BCG therapy (Yates et al, Eur Urol 62 (2012), 1088-1096). The earlier the BCG failure, the more probable is the failure of a second BCG cycle (Gallagher et al., Urology 71 (2008), 297-301).

A second-line conventional BCG immunotherapy after initial BCG failure in non-muscle-invasive bladder cancer, however, was found to be not effective (Di Lorenzo et al., Cancer 2010, doi: 10.1002/cncr.24914). 87.5% patients failed to respond to BCG re-induction at one year. 37.5% of the patients had to undergo cystectomy and 40% underwent radiation therapy plus systemic chemotherapy after one year. The poor outcome in these patients failing to respond to conventional BCG therapy reflects the unmet medical need for improved bladder-sparing treatments after conventional BCG or other intravesical treatment failure. Thus, better treatment options are not only needed for a first-line therapy but also for patients with recurrence after a first course of standard BCG therapy.

Thus, it was an object of the present invention to provide an improved treatment for patients failing to respond to first-line conventional BCG therapy as these patients are at high risk of cancer progression. An improved treatment of these high-risk patients will increase bladder preservation rates and consequently improve quality of life and decrease health costs.

A recombinant BCG strain expressing a phagolysosomal escape domain is described in WO 99/101496, the content of which is herein incorporated by reference. The phagolysosomal escape domain enables the strain to escape from the phagosome of infected host cells by perforating the membrane of the phagosome. In order to provide an acidic phagosomal pH for optimal phagolysosomal escape activity, a urease-deficient recombinant strain was developed. This strain is disclosed in WO 2004/094469, the content of which is herein incorporated.

WO 2012/085101, the content of which is herein incorporated, discloses that a recombinant BCG strain expressing membrane-perforating listeriolysin (Hly) of Listeria monocytogenes and devoid of urease C induces superior protection against aerogenic challenge with Mycobacterium tuberculosis (Mtb) as compared to parental BCG in a preclinical model. Further, it is shown that both the recombinant and the parenteral strain induce marked Th1 immune responses, whilst only the recombinant BCG strain elicits are profound Th17 response in addition.

WO 2016/177717 discloses that a recombinant urease-deficient and listeriolysin-expressing recombinant BCG strain induces a superior immune response compared to the parenteral BCG in an animal model. Further, the start of a human clinical phase I/II trial using recombinant BCG as an immunotherapeutic agent in patients after a first standard BCG therapy is reported.

Despite the discouraging results of Di Lorenzo et al., supra, using conventional BCG therapy as a second-line treatment, the present inventors have surprisingly observed a high overall survival and a low disease recurrence rate even 3 or 4 years after the start of therapy using a urease-deficient and listeriolysin-expressing recombinant BCG as a second-line BCG immunotherapy, particularly after initial BCG failure in non-muscle-invasive bladder cancer.

A first aspect of the present invention relates to a recombinant Mycobacterium cell, which comprises a recombinant nucleic acid molecule encoding a fusion polypeptide comprising:
(a) a domain capable of eliciting an immune response, and
(b) a phagolysosomal escape domain
   for use in a method, wherein the recombinant Mycobacterium cell is administered as an immunotherapeutic agent to a subject, e.g., a human subject suffering from bladder carcinoma, particularly from non-muscle-invasive bladder carcinoma as a second-line therapy.

Accordingly, the present invention further relates to a method for the immunotherapeutic treatment of bladder carcinoma, particularly of non-muscle-invasive bladder carcinoma in a subject, particularly in a human subject,

comprising administering to said subject as a second-line therapy a recombinant Mycobacterium cell, which comprises a recombinant nucleic acid molecule encoding a fusion polypeptide comprising:
(a) a domain capable of eliciting an immune response, and
(b) a phagolysosomal escape domain.

A further aspect of the present invention relates to a recombinant Mycobacterium cell, which comprises a recombinant nucleic acid molecule encoding a fusion polypeptide comprising:
(a) a domain capable of eliciting an immune response, and
(b) a phagolysosomal escape domain
   for use in the immunotherapeutic treatment of bladder carcinoma wherein the recombinant Mycobacterium cell is administered to a subject suffering from bladder carcinoma, particularly from non-muscle-invasive bladder carcinoma, more particularly from recurrent bladder carcinoma as a second-line therapy.

Accordingly, the present invention further relates to a method for the immunotherapeutic treatment of bladder carcinoma, particularly of non-muscle-invasive bladder carcinoma, more particularly from recurrent bladder carcinoma in a subject, particularly in a human subject,

comprising administering to said subject as a second-line therapy a recombinant Mycobacterium cell, which comprises a recombinant nucleic acid molecule encoding a fusion polypeptide comprising:
(a) a domain capable of eliciting an immune response, and
(b) a phagolysosomal escape domain.

A further aspect of the present invention is a recombinant Mycobacterium cell which comprises a recombinant nucleic acid molecule encoding a fusion polypeptide comprising:
(a) a domain capable of eliciting an immune response, and
(b) a phagolysosomal escape domain
   for use as an immunotherapeutic agent in the treatment of bladder carcinoma, in particular recurrent bladder carcinoma, wherein the subject to be treated has relapsed and/or has progressed after a first treatment of bladder carcinoma, particularly after a first treatment with conventional BCG therapy.

Accordingly, the present invention further relates to a method for the immunotherapy of bladder carcinoma in a subject in need thereof, particularly of non-muscle-invasive bladder carcinoma, more particularly from recurrent bladder carcinoma, wherein the subject has relapsed and/or has progressed after a first treatment of bladder carcinoma, particularly after a first treatment with conventional BCG therapy,

comprising administering to said subject a recombinant Mycobacterium cell, which comprises a recombinant nucleic acid molecule encoding a fusion polypeptide comprising:
(a) a domain capable of eliciting an immune response, and
(b) a phagolysosomal escape domain.

A further aspect of the present invention relates to a recombinant Mycobacterium cell, which comprises a recombinant nucleic acid molecule encoding a fusion polypeptide comprising:
(a) a domain capable of eliciting an immune response, and
(b) a phagolysosomal escape domain
   for use in a method, wherein the recombinant Mycobacterium cell is administered as an immunotherapeutic agent to a subject suffering from bladder carcinoma, particularly from non-muscle-invasive bladder carcinoma, more particularly from recurrent bladder carcinoma, wherein disease recurrence is inhibited for a time period of at least 1 year, particularly for a time period of at least 2 years, of at least 3 years, or of at least 4 years.

Accordingly, the present invention further relates to a method for the immunotherapeutic treatment of bladder carcinoma, particularly of non-muscle-invasive bladder carcinoma, more particularly from recurrent bladder carcinoma in a subject, particularly in a human subject, comprising administering to said subject a recombinant Mycobacterium cell, which comprises a recombinant nucleic acid molecule encoding a fusion polypeptide comprising:
(a) a domain capable of eliciting an immune response, and
(b) a phagolysosomal escape domain,
   wherein disease recurrence is inhibited for a time period of at least 1 year, particularly for a time period of at least 2 years, of at least 3 years, or of at least 4 years.

A still further aspect of the present invention relates to a recombinant Mycobacterium cell, which comprises a recombinant nucleic acid molecule encoding a fusion polypeptide comprising:
(a) a domain capable of eliciting an immune response, and
(b) a phagolysosomal escape domain
   for use in a method, wherein the recombinant Mycobacterium cell is administered as an immunotherapeutic agent to a subject suffering from bladder carcinoma, particularly from non-muscle-invasive bladder carcinoma, more particularly from recurrent bladder carcinoma wherein overall survival is increased in a time period of at least 1 year, particularly in a time period of at least 2 years, of at least 3 years, or of at least 4 years.

Accordingly, the present invention further relates to a method for the immunotherapeutic treatment of bladder carcinoma, particularly of non-muscle-invasive bladder carcinoma, more particularly from recurrent bladder carcinoma in a subject, particularly in a human subject, comprising administering to said subject a recombinant Mycobacterium cell, which comprises a recombinant nucleic acid molecule encoding a fusion polypeptide comprising:
(a) a domain capable of eliciting an immune response, and
(b) a phagolysosomal escape domain,
   wherein overall survival is increased in a time period of at least 1 year, particularly in a time period of at least 2 years, of at least 3 years, or of at least 4 years.

A still further aspect of the present invention relates to a recombinant Mycobacterium cell, which comprises a recombinant nucleic acid molecule encoding a fusion polypeptide comprising:
(a) a domain capable of eliciting an immune response, and
(b) a phagolysosomal escape domain
   for use in a method for improving the life quality of a subject suffering from bladder carcinoma, particularly from non-muscle-invasive bladder carcinoma, more particularly from recurrent bladder carcinoma, wherein the recombinant Mycobacterium cell is administered as an immunotherapeutic agent thereby reducing or avoiding cystectomy in said subject.

Accordingly, the present invention further relates to a method for improving the life quality of a subject suffering from bladder carcinoma, particularly from non-muscle-invasive bladder carcinoma, more particularly from recurrent bladder carcinoma in a subject, particularly in a human subject,

comprising administering to said subject a recombinant Mycobacterium cell, which comprises a recombinant nucleic acid molecule encoding a fusion polypeptide comprising:
(a) a domain capable of eliciting an immune response, and
(b) a phagolysosomal escape domain,
   and avoiding or reducing the need of cystectomy in said subject.

It should be noted, that two or more of the features of the above-indicated aspects may be combined with each other.

Thus, the present invention relates to a second-line therapy with rBCG in combination with at least one of:
treatment of a subject who has relapsed and/or has progressed after a first treatment of bladder carcinoma, particularly after a first treatment with conventional BCG therapy,
an inhibition of disease recurrence for a time period of at least 1 year, particularly for a time period of at least 2 years or of at least 3 years, or of at least 4 years,
an increase of overall survival to a time period of at least 1 year, particularly for a time period of at least 2 years, of at least 3 years, or of at least 4 years,
and
an improvement of life quality comprising reducing or the need of cystectomy.

Further, the present invention relates to an improvement of life quality comprising reducing or the need of cystectomy in combination with at least one of:
a second-line therapy for a subject having a disease recurrence and/or disease progression after a failed first-line therapy,
an inhibition of disease recurrence for a time period of at least 1 year, particularly for a time period of at least 2 years, of at least 3 years, or of at least 4 years,
an increase of overall survival to a time period of at least 1 year, and particularly for a time period of at least 2 years, of at least 3 years, or of at least 4 years.

In a particular embodiment, the present invention relates to a second-line therapy in combination with:
an inhibition of disease recurrence for a time period of at least 1 year, particularly for a time period of at least 2 years, of at least 3 years, or of at least 4 years,
an increase of overall survival to a time period of at least 1 year, particularly for a time period of at least 2 years, of at least 3 years, or of at least 4 years,
and optionally an improvement of life quality comprising reducing or the need of cystectomy.

The present inventors have found that a second-line immunotherapy comprising administration of a recombinant Mycobacterium cell as described herein was highly effective in reducing disease occurrence and increasing overall survival. In this context, the term “second-line immunotherapy” refers to the treatment of a subject, particularly a human subject, after failure of a first-line therapy. The term “failure” particularly means disease recurrence and/or disease progression including progression of the primary tumor and/or formation of metastases after first-line therapy. More particularly, the term “failure” means disease recurrence. The term “first-line therapy” particularly refers to a first-line therapy including an immunotherapy, and more particularly to a first-line immunotherapy comprising administration of standard BCG without a phagolysosomal escape domain as described herein, such as previously approved standard BCG as described in Rentsch, C A, Eur Urol 2014, PMID: 24674149. The term “second-line immunotherapy” comprises administration of a recombinant Mycobacterium cell as described herein after a single “first-line therapy” cycle or after multiple, e.g., 2, 3 or more “first-line therapy” cycles, particularly a single cycle or multiple cycles comprising administration of BCG without a phagolysosomal escape domain.

According to a preferred embodiment of the invention the recombinant Mycobacterium cell is administered as a second-line treatment. According to such embodiments, the individual to be treated may have received a first treatment, in particular of bladder carcinoma treatment, selected from the group of standard BCG treatment, chemotherapy including systemic and/or intravesical chemotherapy, bladder surgery, radiation and any combination thereof. For example, the first-line treatment may include cisplatin-based chemotherapy, in particular cisplatin-based chemotherapy followed by bladder surgery and/or radiation therapy, concomitant chemotherapy and standard BCG. Preferably, the individual to be treated has received standard BCG treatment as a first treatment of bladder carcinoma and/or underwent cystectomy or another local treatment or systemic chemotherapy.

The subject to be treated according to the present invention is suffering from bladder carcinoma, particularly non-muscle invasive bladder carcinoma (NMIBC) including carcinoma in situ (CIS). In particular embodiments, the subject is suffering from recurrent NMBIC. In some embodiments, the subject has a recurrent high-grade NMIBC for progression (e.g., score 7-23) based on the European Organization for Research and Treatment. In further embodiments, the subject has undergone to a transurethral resection of the bladder tumor (TURBT) before therapy start. In some embodiments, the subject has a tumor-free bladder state at therapy start, e.g., as confirmed by transurethral resection of the bladder (TURB) and biopsy.

In particular embodiments, the subject is a patient with a high grade NMIBC tumor present at 6 months or both at 3 months and 6 months after start of a previous cycle of standard BCG therapy and worsening of the disease under standard BCG therapy including at least one of higher number of recurrences, higher tumor category or higher grade, and appearances of CIS, including low grade tumors, optionally in spite of an initial response.

According to an especially preferred embodiment, the subject is a smoker.

Administration of a recombinant Mycobacterium cell as described herein as a second-line therapy is highly effective in reducing disease occurrence for an extended time period, e.g., a time period of at least 1 year, particularly for a time period of at least 2 years, of at least 3 years or of at least 4 years after treatment start. 3 years after treatment start, the median time of disease recurrence is already 1.3 years and will probably increase further. At this time, no tumor recurrence was observed in about 49% of the patients.

Thus, the therapy of the present invention provides a disease-free time period of at least 1 year, particularly of at least 2 years, of at least 3 years, or of at least 4 years for at least 30% of the patients and particularly for at least 45% of the patients.

Notably, in the time period between 2 and 3 years or between 2 and 4 years after treatment start, no new disease recurrence was observed. Thus, the treatment is highly efficient in stopping disease recurrence in a sub-group of patients, who did not suffer from disease recurrence in a time period up to 2 years after treatment start.

In contrast thereto, administration of a standard BCG as a second-line therapy was not effective at all as described by Di Lorenzo et al. supra, since 2 years after treatment start in only less than 5% of the patients no tumor recurrence was observed.

This high clinical efficiency of the therapy of the present invention was found without the occurrence of clinically significant adverse events. Thus, the treatment translates into an overall survival benefit for the patients.

Thus, administration of a recombinant Mycobacterium cell as described herein is highly effective in increasing the overall survival in an extended time period, e.g., a time period of at least 1 year, particularly in a time period of at least 2 years, of at least 3 years or of at least 4 years. 3 years after start of the treatment, the overall survival rate was between 70% and 80%.

In particular embodiments, the therapy of the invention is suitable in avoiding cystectomy in a large number of patients resulting in a high improvement of life quality. For example, cystectomy may be avoided for a time period of at least a 1 year, particularly for a time period of at least 2 years, of at least 3 years or of at least 4 years in at least 30% of the patients and particularly for at least 45% of the patients.

The immunotherapeutic agent is a live recombinant Mycobacterium cell, which comprises a recombinant nucleic acid molecule encoding a fusion polypeptide comprising (a) a domain capable of eliciting an immune response and (b) a phagolysosomal escape domain. The domain capable of eliciting an immune response is preferably an immunogenic peptide or polypeptide from a pathogen or an immunogenic fragment thereof.

The Mycobacterium cell is preferably an M. bovis cell, an M. tuberculosis cell, particularly an attenuated M. tuberculosis cell or other Mycobacteria, e.g. M. microti, M. smegmatis, M. canettii, M. marinum or M. fortuitum. More preferably, the cell is an attenuated recombinant M. bovis (BCG) cell, particularly an M. bovis BCG cell, more particularly a recombinant M. bovis BCG cell from strain Danish subtype Prague (Brosch et al., Proc. Natl. Acad. Sci. USA, 104 (2007), 5396-5601). In an especially preferred embodiment, the Mycobacterium cell is recombinant urease-deficient. In an especially preferred embodiment, the ureC sequence of the Mycobacterium cell is inactivated (AUrec), e.g., by constructing a suicide vector containing a ureC gene disrupted by a selection marker gene, e.g., the hygromycin gene, transforming the target cell with the vector and screening for selection marker-positive cells having a urease negative phenotype. In an even more preferred embodiment, the selection marker gene, i.e., the hygromycin gene, is subsequently inactivated. In this embodiment, the cell is a selection marker-free recombinant Mycobacterium cell. Most preferably, the cell is selection marker-free recombinant BCG strain Danish subtype Prague characterized as recombinant BCG ΔUrec::Hly+.

The domain capable of eliciting an immune response is preferably selected from immunogenic peptides or polypeptides from M. bovis, M. tuberculosis or M. leprae or from immunogenic fragments thereof having a length of at least 6, preferably at least 8 amino acids, more preferably at least 9 amino acids and e.g., up to 20 amino acids. Specific examples for suitable antigens are Ag85B (p30) from M. tuberculosis, Ag85B (α-antigen) from M. bovis BCG, Ag85A from M. tuberculosis and ESAT-6 from M. tuberculosis and fragments thereof. In other embodiments, the domain capable of eliciting an immune response is selected from non-Mycobacterium polypeptides.

More preferably, the immunogenic domain is derived from the antigen Ag85B. Most preferably, the immunogenic domain comprises the sequence from aa. 41 to aa.51 in SEQ ID No.2.

The recombinant nucleic acid molecule further comprises a phagolysosomal escape domain, i.e., a polypeptide domain which provides for an escape of the fusion polypeptide from the phagolysosome into the cytosol of mammalian cells. Preferably, the phagolysosomal escape domain is a Listeria phagolysosomal escape domain, which is described in U.S. Pat. No. 5,733,151, herein incorporated by reference. More preferably, the phagolysosomal escape domain is derived from the listeriolysin gene (Hly) of L. monocytogenes. Most preferably, the phagolysosomal domain is encoded by a nucleic acid molecule selected from: (a) a nucleotide sequence comprising nucleotides 211-1722 as shown in SEQ ID No.1, (b) a nucleotide sequence which encodes the same amino acid sequence as the sequence from (a), and (c) a nucleotide sequence hybridizing under stringent conditions with the sequence from (a) or (b).

Apart from the nucleotide sequence depicted in SEQ ID No.1 the present invention also comprises nucleic acid sequences hybridizing therewith. In the present invention, the term “hybridization” is used as defined in Sambrook et al. (Molecular Cloning. A laboratory manual, Cold Spring Harbor Laboratory Press (1989), 1.101-1.104). In accordance with the present invention the term “hybridization” is used if a positive hybridization signal can still be observed after washing for one hour with 1×SSC and 0.1% SDS at 55° C., preferably at 62° C. and more preferably at 68° C., particularly for 1 hour in 0.2×SSC and 0.1% SDS at 55° C., preferably at 62° C. and more preferably at 68° C. A sequence hybridizing with a nucleotide sequence as per SEQ ID No.1 under such washing conditions is a phagolysosomal escape domain encoding nucleotide sequence preferred by the subject invention.

A nucleotide sequence encoding a phagolysosomal escape domain as described above may be directly obtained from a Listeria organism or from any recombinant source e.g., a recombinant E. coli cell containing the corresponding Listeria nucleic acid molecule, or a variant thereof as described above.

Preferably, the recombinant nucleic acid molecule encoding for a fusion polypeptide contains a signal peptide encoding sequence. More preferably, the signal sequence is a signal sequence active in Mycobacteria, preferably in M. bovis, e.g., a native M. bovis signal sequence. A preferred example of a suitable signal sequence is the nucleotide sequence coding for the Ag85B signal peptide, which is depicted in SEQ ID No.1 from nucleotide 1 to 120.

Further, it is preferred that a peptide linker be provided between the immunogenic domain and the phagolysosomal escape domain. Preferably, said peptide linker has a length of from 5 to 50 amino acids. More preferably, a sequence encoding a linker as shown in SEQ ID No.1 from nucleotide 154 to 210 or a sequence corresponding thereto as regards the degeneration of the genetic code.

The nucleic acid may be located on a recombinant vector. Preferably, the recombinant vector is a prokaryotic vector, i.e., a vector containing elements for replication or/and genomic integration in prokaryotic cells. Preferably, the recombinant vector carries the nucleic acid molecule of the present invention operatively linked with an expression control sequence. The expression control sequence is preferably an expression control sequence active in Mycobacteria, particularly in M. bovis. The vector can be an extrachromosomal vector or a vector suitable for integration into the chromosome. Examples of such vectors are known to the man skilled in the art and, for instance, given in Sambrook et al. supra.

The immunotherapeutic agent of the present invention is suitable for the treatment of bladder carcinoma, e.g., non-invasive bladder carcinoma, e.g. non-invasive papillary carcinoma in situ (Tₐ), non-invasive carcinoma in situ (T_{cis}), tumor invading subepithelial connective tissue (T₁), tumor invading superficial muscle (inner half) (T₂a), tumor invading deep muscle (outer half) (T_{2b}), tumor invading perivesical tissue (T₃ including T₃a and T_{3b}), tumor invading prostate, uterus or vagina (T₄ₐ), and tumor invading pelvic wall or abdominal wall (T_{4b}). Particularly, the tumor is a superficial tumor or carcinoma in situ (T_{cis}), non-invasive papillary carcinoma (Tₐ), or a tumor invading subepithelial connective tissue (T₁). The immunotherapeutic treatment is suitable in the treatment of primary bladder carcinoma and/or in the treatment of recurring bladder carcinoma.

The immunotherapeutic agent is locally administered to the tumor site, i.e., to the site of a primary tumor before surgery or after surgery and optionally after chemotherapy. For the treatment of urothelial bladder carcinoma, the agent is preferably administered by vesicular instillation into the urinary bladder.

The immunotherapeutic agent is administered to the subject to be treated in an effective dose. For a human subject, the dose for an administration may be about 10⁶ to 10¹⁰ viable units (CFU), e.g., about 10⁷ to 5×10⁹ or 10⁸ to 3×10⁹ viable units. Preferably, the dose for an administration is about 2×10⁹viable units (CFU). Preferably, the immunotherapeutic agent is administered several times, e.g., at least 3 times or at least 5 times up to 30 times, particularly about 15 times, at predetermined times during the treatment.

The immunotherapeutic agent is usually provided as a pharmaceutical preparation, which comprises the recombinant Mycobacterial cell in solid form, e.g., a lyophilized or cryoconserved preparation, which is reconstituted with a suitable liquid carrier before use. Alternatively, the preparation may be provided in liquid form, e.g., as suspension.

In one embodiment, the immunotherapeutic agent of the invention is administered for the treatment of carcinoma in situ. A standard schedule may comprise weekly administration of the agent for at least 4, e.g., 4, 5, 6, 7 or 8 weeks as an induction therapy. The induction therapy should not start until 2-3 weeks after primary tumor surgery. After a treatment-free interval of, e.g., 4 weeks, administration may continue using maintenance therapy for at least 6 months or at least 1 year.

In a further embodiment, the immunotherapeutic agent is administered in an induction therapy in the prophylactic treatment of tumor recurrence. In this embodiment, therapy may start about 2-3 weeks after biopsy of the tumor site and be repeated, e.g., at weekly intervals for at least 4, e.g., 4, 5, 6, 7 or 8 weeks. In intermediate and high-risk tumors this may be followed by maintenance therapy.

Maintenance therapy may comprise long-term therapy, e.g., 6, 9 or 12 months therapy or even longer with treatments at monthly intervals. Alternatively, maintenance therapy may comprise 2, 3 or 4 administrations at weekly intervals, at month 3, 6, 12, 18, 24, 30 and 36.

In a particular embodiment, the immunotherapeutic agent, particularly recombinant BCG AUrec::Hly+, is used for the treatment of non-muscle invasive bladder cancer in patients with recurrence after standard BCG therapy. The immunotherapeutic agent is administered into the bladder according to a schedule involving weekly instillations during an induction phase with e.g., 6 weekly instillations, a first maintenance phase after about 3 months with e.g., 3 weekly instillations, a second maintenance phase after about 6 months with e.g., 3 instillations and a third maintenance phase after about 12 months with e.g., 3 instillations.

The administration as immunotherapeutic agent of the recombinant Mycobacterium cell as described above may be combined with further anti-tumor therapy, e.g., radiation and/or chemotherapy. Further, the immunotherapy as described above may be combined with a non-tumor site specific administration of the recombinant Mycobacterium cell in order to provide a general stimulation of the immune system. This non-site specific administration may be effected as described in WO 2012/085101, e.g., before surgery of the primary tumor. In this case, the agent is preferably administered to a human subject in a dose of about 1-10×10⁵, preferably about 2-8×10⁵ cells. The agent is preferably administered as a single dose, e.g., by injection. Subcutaneous injection is preferred. Further, it is preferred to administer the agent without adjuvant.

Further, the invention is described in more detail by the following Figures and Examples 1 and 2.

The immunotherapeutic agent “rBCG” used in these examples is recombinant M. bovis (BCG) Danish subtype Prague with an inactivated ureC sequence (AUrec) and without functional selection marker gene which expresses an Ag85B/Hly fusion protein as shown in SEQ ID No.2 (Hly+).

### EXAMPLE 1

Phase I/II open label clinical trial assessing safety and efficacy of intravesical instillation of recombinant BCG (rBCG) in human patients with recurrent non-muscle invasive bladder cancer after standard BCG therapy

### 1.1 Phase I

Phase I was conducted to determine safety, tolerability, and the recommended phase II dose of intravesical rBCG instillations in patients with recurrence of non-muscle-invasive bladder cancer after TURB and standard BCG therapy.

### 1.2 Phase II

Phase II was conducted to investigate the efficacy, safety, tolerability and immunogenicity of intravesical rBCG instillations in patients with recurrence of non-muscle-invasive bladder cancer after TURB in standard BCG therapy.

1.3 Clinical protocol

rBCG was administered into bladder in 15 weekly instillations (induction phase: instillations 1-6; maintenance 3 months: instillations 7-9; maintenance 6 months: instillations 10-12, maintenance 12 months: instillations 13-15).

The primary endpoint of the phase I was dose limiting toxicity (DLT) of intravesical rBCG instillations in patients with recurrence after standard BCG therapy in non-muscle invasive bladder cancer (NMIBC). The DLT period corresponds to 3 instillations plus 1 week and covers acute toxicities induced by treatment. Patients were treated in two cohorts of three, following the rules of a 3+3 design (dose de-escalation rules: if patients treated at dose level 1 show signs of DLT, dose of instilled rBCG will be reduced to level −1, which is 10 times lower than level 1).

The dose levels were as follows:
Dose level 1: 1-19.2×10⁸CFUs of rBCG
Dose level −1: 1-19.2×10⁷CFUs of rBCG

### 1.4 Selection of Patients

### 1.4.1 Inclusion Criteria

Histologically confirmed diagnosis of recurrent NMIBC including repeat TURB confirming tumor-free state of the bladder; for patients with pure CIS no repeat TURB is necessary.
Negative cytology before start of treatment except for patients with concomitant CIS.
Planned treatment start is 2-6 weeks after last TURB.
One previous cycle of intravesical BCG (induction phase with at least 5 instillations ±maintenance) not more than 5 years ago for NMIBC.
Recurrent high-grade NMIBC for progression (progression score 7-23) based on the European Organization for Research and Treatment of Cancer scoring system, failing BCG therapy (Babjuk M, Eur Urol 2008 PMID 18468779), for whom radical cystectomy or re-induction with standard BCG is indicated.

This particularly includes patients with high grade NMBIC tumor(s) present at both 3 and 6 months after start of the previous cycle of BCG therapy, and any worsening of the disease under BCG treatment such as higher number of recurrences, higher tumor category or higher grade, or appearances of CIS, including low grade tumors, in spite of an initial response.

### 1.4.2 Exclusion Criteria

Current or previous ≥T2 urothelial carcinoma (UC) of the urinary bladder.
Concomitant UC of upper urinary tract, prostate or urethra, small cell carcinoma of the bladder, micropapillary urothelial carcinoma, pure squamous cell carcinoma of the bladder, pure adenocarcinoma, lympho-vascular invasion in the TURB specimen.
Previous or current evidence of UC in a bladder diverticulum.
Evidence of metastatic disease in the (thoraco-) abdominal CT scan at registration.
Bladder surgery or dramatic catheterization or TURB within 2 weeks prior to the start of treatment.
Administration of systemic cytotoxic agents within the past 3 years or administration of repeated cytostatic/cytotoxic drugs by intravesical instillations after EUC recurrence after the first BCG therapy (during the past 5 years).

### 1.5 Current Status

Clinical phase I is completed. Phase II was conducted with dose level 1: 1-19.2×10⁸ CFU of rBCG.

The active agent was administered in 50 ml drug solution containing dextran, glucose, 0.9% sodium chloride, 0.025% Tween 80 and water for injection.

The follow-up data for recurrence in the bladder status of all evaluable phase II patients at 3 years are shown in Table 1. 20 patients experienced a confirmed event and 1 patient an unconfirmed event. Ten patients remained alive and on follow-up for this endpoint.

40 patients were included, all with high-grade tumors as specified above. The rate of CIS was high (68%). Recurrent-free state in the bladder (recurrence-free survival at 60 weeks was 49.3% and at three years 43.7% with a 95% confidence interval [26.9%, 59.4%] as shown in FIG. 1.

The treatment was safe and well tolerated, with only 5% of patients unable to tolerate adequate induction therapy. No dose-limiting toxicity occurred and no grade 3 or 4 adverse events were observed.

Overall survival after a median follow-up time of 2.9 years is shown in FIG. 2. Until this analysis, 7 patients died, 9 patients were lost to follow-up and 24 patients remained on follow-up. Only two patients (8%) were required to undergo cystectomy. 4 patients were required to switch to chemotherapy.

### EXAMPLE 2

Phase I/II open label clinical trial assessing safety and efficacy of intravesical instillation of recombinant BCG (rBCG) in human patients with recurrent non-muscle invasive bladder cancer after conventional BCG therapy

### 2.1 Patients and Methods

2.1.1 Study design and participants

The trial was designed as a multicentre, open-label, single-arm, phase I/II study and conducted in compliance with the current version of the declaration of Helsinki, the ICH-GCP, and with national legal and regulatory requirements. Written informed consent was obtained from all patients prior to enrolment.

Eligible patients had recurrent NMIBC with intermediate and high-risk for progression (score 7-23 based on the European Organization for Research and Treatment of Cancer scoring system) after conventional BCG therapy.

Persistent T1 disease or high-grade non-muscle invasive tumour(s) present at both 3 and 6 months after start of the previous cycle of BCG therapy, or any worsening of the disease under BCG treatment, such as a higher number of recurrences, higher tumour category or higher grade, or appearance of carcinoma in situ (CIS), in spite of an initial response were required (EAU 2008 guideline definition). Prior therapy was defined as one previous cycle of intravesical BCG (induction phase ≥5 instillations ±BCG maintenance). For inclusion, histologically confirmed diagnosis of recurrent NMIBC and a repeat transurethral resection of the bladder (TURB) confirming a tumour-free state according to current pathology guidelines were mandatory. In patients presenting with CIS, selective upper tract cytology and biopsies of the prostatic urethra were recommended. At the start of the study treatment, bladder wash cytology had to be negative, except for patients with pure or concomitant CIS and imaging without evidence of metastatic disease.

Exclusion criteria were stage ≥T2 urothelial carcinoma of the bladder, concomitant urothelial carcinoma of the upper urinary tract, the non-prostatic urethra, or evidence of metastatic disease (https://clinicaltrials.qov/ct2/show/NCT02371447).

rBCG was provided as a formulated lyophilized powder of 1-19.2×10⁸ CFU/vial of live Mycobacterium bovis BCGΔureC::hly (SIIPL, Serum Institute of India Private Limited, Pune, India) and reconstituted in 50 ml of 0.9% saline water for intravesical application. Patients were scheduled for standard treatment of 6 weekly intravesical instillations followed by maintenance of 1 year (3 instillations at 3, 6 and 12 months after first instillation).

The primary endpoint of the phase II part was defined as the recurrence-free rate (RFR) in the bladder 60 weeks after registration. In case of suspected recurrence due to positive cytology or visual detection of a tumour in the bladder at cystoscopy, histological proof of the recurrence was required. A recurrence in the bladder associated with cancer in the prostatic urethra or evidence for cancer in the upper urinary tract were not considered as a recurrence in the bladder. Predefined secondary endpoints included time from trial registration to recurrence in the bladder, time to progression, overall survival (OS), AEs, tolerability, and QoL. Progression was defined as progression to muscle-invasive bladder cancer or progression to metastatic disease. OS was calculated from registration until death from any cause. Tolerability was defined as the proportion of patients finishing five instillations of induction within 12 weeks after treatment start.

### 2.1.2 Statistical Analyses

For sample size calculation, we estimated the RFR from one of the few prospective randomized European trials comparing BCG re-exposure versus gemcitabine. In this trial, the group receiving BCG had an estimated RFR and progression-free rate 60 weeks after the last TURB of 15% and 62.5%, respectively. A single-stage design was used with H0: RFR at 60 weeks ≤15%; and H1: RFR at 60 weeks ≤30%. For a significance level of 10% and a power of 80%, 37 evaluable patients for phase II were required based on the Kaplan-Meier estimator evaluated 60 weeks after registration (R package OptInterim). The 6 patients from the phase I part were included for the phase II part. To account for non-evaluable patients, the sample size was increased to 39 patients.

### 2.2 Results

### 2.2.1 Patients

From September 2015 to April 2018, 42 patients were enrolled into the trial. A total of 16 sites were activated and 14 sites actively recruited patients. Two patients had to be excluded from efficacy analysis because of major inclusion criteria violation but were kept in for safety/ITT analysis. Forty patients defined the full analysis set (FAS) and the per protocol population (PP) consisted of 22 patients. Definitions of analysed populations and detailed reasons for exclusions are given in FIG. 3.

The detailed tumour characteristics of the primary NMIBC occurrences of the patient cohort are given in Table 2 and the detailed patient and tumour characteristics of the FAS in Table 3. The median risk for progression of the recurrent NMIBC increased by 7 risk points as compared to the primary occurrence. A history of chemical exposure as a risk factor for bladder cancer was identified in 3 patients, while 21 patients (52.5%) had a smoking history with a median (range) of 38 (4.0-99.0) pack-years. Concomitant or pure CIS was present in 27 (67.5%) patients. Fourteen patients (35%) received BCG maintenance therapy. The median progression score was 16 (7-20) for those patients with and 16 (7-19) for those without previous BCG maintenance.

All patients enrolled in the trial started trial treatment. Thirty-two patients (80%) started maintenance therapy. Fifteen patients (37.5%) received all scheduled instillations of induction and maintenance therapy. Detailed reasons for stopping treatment during the trial are given in FIG. 3. Median follow-up at the time of analysis was 74.4 (12.3-140.9) weeks.

### 2.2.2 Primary Endpoint

After rBCG induction and maintenance therapy, 49.3% [95% CI 32.1, 64.4] of the patients were without evidence of recurrence in the bladder 60 weeks after trial registration. After a median follow-up of 2.9 years, the RFR remained at 47.4% [30.4%, 62.6%] and 43.7% [26.9%, 59.4%]2 and 3 years after trial registration, respectively. The median time from trial registration to recurrence in the bladder was 54.1 weeks [95% CI 38.4 weeks](FIG. 4). The RFR in the bladder for the ITT and PP populations 60 weeks after registration was 48.0% [95% CI 31.1, 63.0] and 40.8% [95% CI 19.9, 60.9], respectively. If accounting for all potential recurrences including histologically unconfirmed but clinically evident extravesical recurrences in the FAS population, the 60-week RFR reached 47.6% [95% CI 30.8, 62.6]. Out of the patients with CIS (n=27), 15 (55.6%; 95% CI [35.3%, 74.5%]) had a complete response (CR) at 12 weeks after treatment start. The median duration of response (DOR) for these 15 patients was 1.1 years (95% CI [0.4 years-not reached]). Further subgroup evaluations related to the primary endpoint are given in Table 3.

### 2.2.3 Secondary Endpoints

Seven patients from the FAS experienced progressive disease (median time to progression not reached). Three patients showed progression to muscle-invasive disease, and 4 patients progressed with metastatic disease (regional lymph nodes n=3, distant metastasis n=1). None of the patients with metastatic disease were found to have concomitant muscle invasive disease in the bladder.

Follow-up treatment for the patients with recurrence after trial treatment is listed in Table 5. Until the time of analysis, 5 patients had died (2 from bladder cancer, 2 from other cancers not known at inclusion, and one from acute respiratory distress syndrome unrelated to the study treatment).

In summary, 60 weeks after trial registration, progression-free rate and OS rate were 76.3% [95% CI 56.4, 88.0] and 92.9% [95% CI 74.3, 98.2], respectively.

### 2.2.4 Adverse Events and Tolerability

Treatment-related AEs are listed in Table 6. The major grade 2 AE term was genitourinary tract infection with common uropathogenic bacteria in one third of the patients. Two patients had to undergo in-hospital antibiotic treatment for urogenital tract infection.

No grade 4 or 5 AEs occurred during the treatment phase. Two of the 42 patients did receive less than five instillations, one because of a BCG-induced systemic inflammatory reaction (BCGitis) due to traumatic catheterisation and one because of patient refusal (FIG. 3).

### 2.2.5 PPD Testing

77.5% of the patients were PPD negative (PPD−) at treatment start (Table 2). Conversion of the PPD test after finishing the rBCG therapy was seen in 7 patients only (PPD- to PPD+: n=5; PPD+ to PPD−: n=2). Fifteen patients (37.5%) did not convert from PPD- to PPD+. In these patients, the RFR in the bladder was 62.9% (Table 4).

### 2.2.6 Quality of Life

All 40 patients in the FAS completed the QoL questionnaire at baseline; 32 of 33 (97%) patients who started the maintenance therapy completed it at the beginning of maintenance therapy and 32 of 40 (77.5%) completed it at the end of treatment. Fourteen patients (45.2%) completed the QoL assessment after the 15 instillations as required by protocol. Patients reported high levels of QoL for the functional scales and a low symptom burden at baseline. Some impairment was reported with regard to urinary symptoms, future worries, and sexual issues. Most of the QoL scales showed stable scores from baseline to the beginning of maintenance therapy. Small improvements were observed for emotional functioning and urinary symptoms. Future worries improved (i.e., patients expressed less worries) to a clinically relevant extent. Almost half (n=13, 49%) of the patients reported a clinically relevant improvement in emotional functioning, whereas approx. one third reported a clinically relevant deterioration in physical well-being (n=10, 30%), global health status/QoL (n=11, 33%), or fatigue (n=10, 30%) during induction therapy. No major changes from baseline to the end of treatment were observed for any of the QoL scales in those patients who completed treatment according to protocol.

### 2.3 Discussion

### 2.3.1 Endpoint Analysis

We have chosen a high-risk population for recurrence and progression with previous conventional BCG exposure to test rBCG. Given the poor outcome of BCG re-exposure in these patients with reported 12 and 24 months RFR of 15% and 3%, respectively, we considered a placebo-controlled trial or a trial comparator with conventional BCG as unethical. With 49.3% RFR in the bladder, this single-arm trial has clearly met the primary endpoint of the predefined RFR >30% 60 weeks after trial registration. This is also true when extending the definition of recurrence to extra-vesical recurrences including histologically unconfirmed clinically evident recurrences in the FAS population, the ITT and the PP population, as well as whether the patients received BCG maintenance or not (Table 3). With a RFR of 47.4% and 43.7% after 2 and 3 years, the treatment response remains stable indicating a potentially promising long-term effect.

After the initiation of this trial, the Food and Drug Administration (FDA) published a new definition for BCG failure and the International Bladder Cancer Group recommended a clinically meaningful initial complete response rate (for CIS) or recurrence-free rate (for papillary tumours) of at least 30% at 12 months for single arm trials in BCG unresponsive patients. According to the FDA definition of BCG failure, 16 patients (40.0%) qualified for BCG unresponsive disease. When applying these more stringent criteria, the 60-week RFR dropped to 24.0% (Table 3).

Further subgroup analysis related to the primary endpoint indicated that smoking status, presence of CIS, and previous BCG maintenance therapy were possibly associated with a worse outcome in this trial, which were, however, not statistically significant-a result again limited by the small sample size (Table 3).

Most of the patients with recurrence underwent cystectomy or other local treatments, and 3 patients underwent systemic chemotherapy (Table 4). While missed metastatic disease at inclusion is a potential explanation for such a fatal development, it cannot be excluded that metastatic disease evolved after TUR and/or under intravesical trial therapy. Notably, none of the patients with metastatic disease were found to have concomitant muscle invasive disease in the bladder.

### 2.3.2 Adverse Events and Tolerability

The most common AE was genitourinary (GU) infection with common bacteria, occurring in one third of the patients. Potential explanations are i) that lubricants containing antiseptics for instillations were not allowed, and ii) that asymptomatic patients with positive urine dipstick had to undergo antibiotic treatment when urine culture was positive. Importantly, the GU infections did not significantly impact on QoL of the patients (not shown). No grade 4 or 5 AE occurred, and tolerability (defined as patients receiving more than 4 instillations during induction) was 95.2%. The treatment can therefore be considered as safe and well tolerated.

### 2.3.3 PPD Testing

Experimental models have suggested that either a PPD+ state prior to the start of treatment or a conversion from PPD- to PPD+ state during treatment might predict response to therapy. In clinical trials, the clinical correlation of the PPD test with the outcome has been conflicted by heterogeneous results, by previous mycobacterial exposure, and different handling in the execution and interpretation of the test itself. In the present study, most patients (77.5%) had a negative PPD test before the start of the treatment with rBCG, although they had been exposed to BCG previously. Unexpectedly, patients with negative PPD test at inclusion showed a 60-week RFR in the bladder of 52.8%, and even without conversion from PPD- to PPD+, the RFR in the bladder remained 62.9% (n=15; Table 3), indicating that a negative PPD test and non-conversion might not be able to predict treatment outcome in this setting.

### 2.3.4 Qol

Patients reported overall stable QoL during induction and maintenance treatment, with small improvements in emotional functioning and future worries. Around 30% of patients reported clinically relevant deteriorations for physical functioning, global QoL, or fatigue during induction therapy. Interestingly, for patients who completed the treatment according to protocol no major changes were observed for any of the QoL scales. Again, the limited sample size without a comparator arm stresses for a cautious interpretation of these QoL results.

If the favorable Qol and tolerability is confirmed in larger studies, rBCG has the potential to decrease the proportion of patients becoming intolerable to BCG treatment and may be combined with other agents such as check-point inhibitors in order to increase efficiency.

### 2.4 Conclusions

The study has met the primary endpoint and demonstrates freedom from recurrence in the bladder after intravesical rBCG treatment in almost half of the patients after one year. rBCG has a promising tolerability, safety, and QoL profile.

### TABLES AND FIGURE LEGENDS

Table 1. Follow-up data for recurrence in the bladder status of all evaluable phase II patients at 3 years.

Table 2. Baseline tumor characteristics of first occurrence of NMIBC (before first conventional BCG treatment).

Table 3. Baseline patient and tumor characteristics of the FAS (full analysis set). WHO: World Health Organisation. BCG: Bacillus Calmette Guerin. CIS: Carcinoma in situ. EORTC: European Organisation for Research and Treatment of Cancer.

Table 4. Subgroup efficacy analysis of the FAS showing recurrence-free rate in the bladder at 60 weeks together with 95% CI. CIS: Carcinoma in situ. BCG: Bacillus Calmette-Guerin. FDA: Food and Drug Administration. PPD: Purified protein derivative.

Table 5. Treatment of patients with recurrence and/or progression after trial treatment. BCG: Bacillus Calmette Guérin. Some patients received more than one treatment. One of the 13 patients undergoing cystectomy received surgery due to chronic bladder infection and not because of cancer recurrence.

*one of these treatments was performed based on a recurrence later than 60 weeks after registration.

Table 6. Treatment associated adverse events (AE) in the treatment phase. BCG: Bacillus Calmette-Guerin. GU: Genito-urinary.

FIG. 1. Kaplan Meier plot for time to recurrence in the bladder after a follow-up time up to 2.9 years.

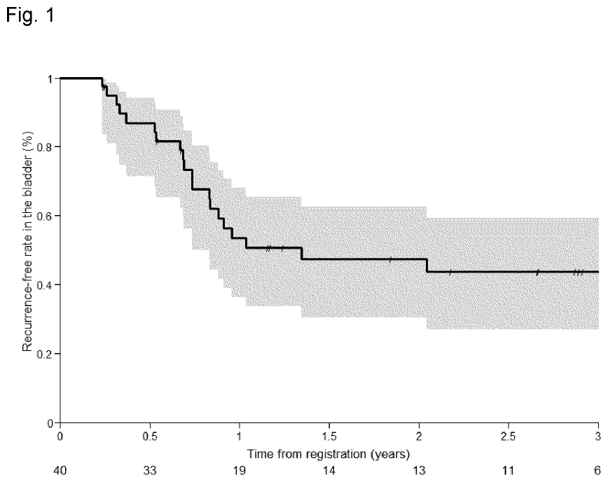

FIG. 2. Overall survival after a follow-up time up to 2.9 years.

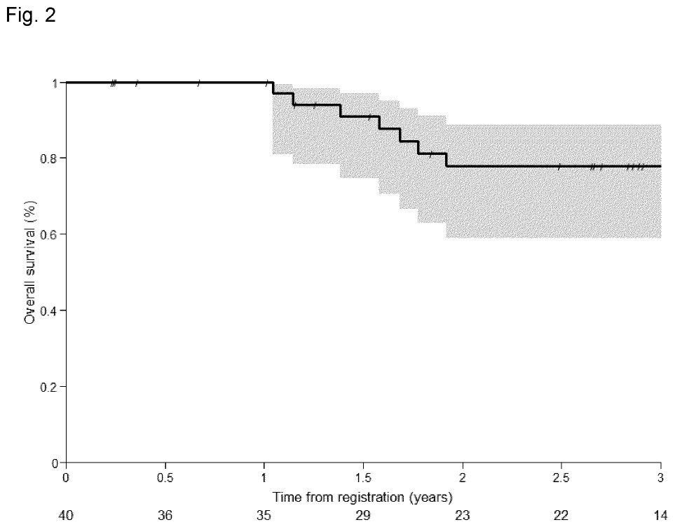

FIG. 3. CONSORT 2010 diagram of SAKK 06/14. BCG: Bacillus Calmette-Guerin. ITT: Intention to treat. FAS: Full analysis set (population). PP: Per-protocol (population). ¹ includes also the 2 exclusions from FAS.

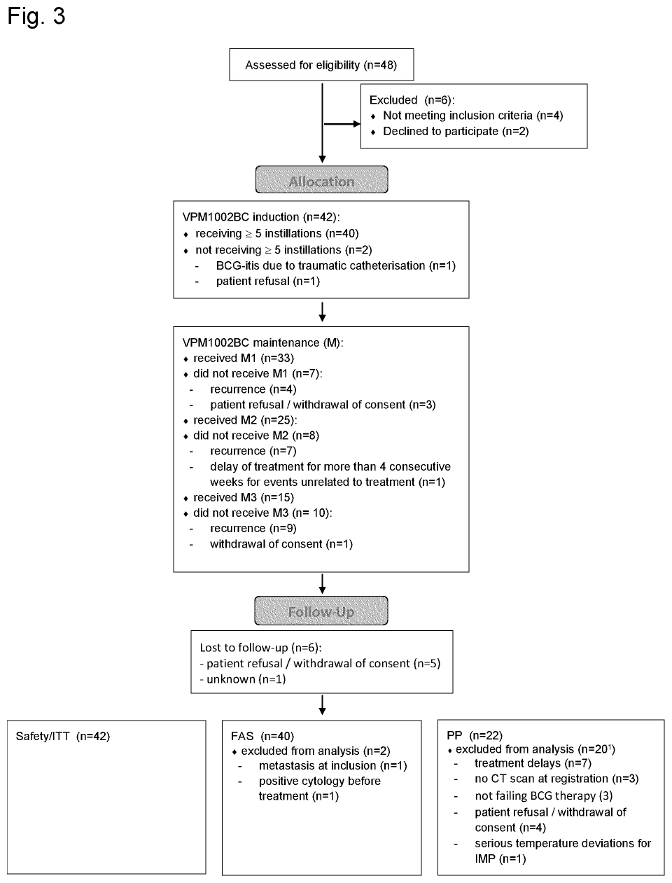

FIG. 4: Kaplan Meier plot for time to recurrence in the bladder for the FAS (follow up to 4 years).

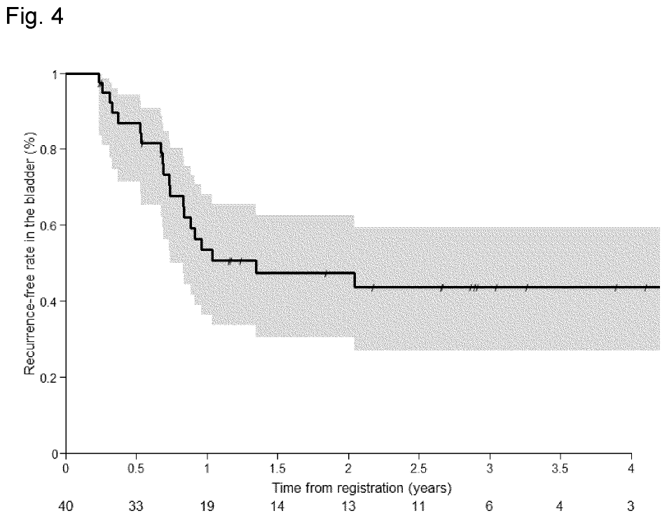

TABLE 1

| | Full |
|---|---|
| | Analysis |
| | Set |
| | (N = 40) |
| Variable | n |
| Recurrence in the bladder status | |
| Recurrence in the bladder | 20 |
| Recurrence in the bladder | 1 |
| (unconfirmed) | |
| Censored at last assessment (alive) | 10 |
| Censored at last assessment (dead) | 1 |
| Censored at last assessment (lost to | 4 |
| follow-up) | |
| Censored at start of new treatment | 2 |
| Censored at cystectomy date | 2 |

TABLE 2

| | FAS |
|---|---|
| Variable | n (%) |
| T classification | |
| T1 | 18 (45.0) |
| T1/CIS | 6 (15.0) |
| Ta | 6 (15.0) |
| Ta/CIS | 5 (12.5) |
| CIS | 5 (12.5) |
| 1973 WHO grading | |
| Grade 2 | 5 (12.5) |
| Grade 3 | 34 (85.0) |
| Unknown | 1 (2.5) |
| 2004 WHO grading | |
| Low-grade urothelial carcinoma | 4 (10.0) |
| High-grade urothelial carcinoma | 35 (87.5) |
| Unknown | 1 (2.5) |
| EORTC progression score | |
| 0 | 1 (2.5) |
| 1-6 | 7 (17.5) |
| 7-13 | 26 (65.0) |
| 14-23 | 6 (15.0) |
| Median (min, max) | 9 (0.0-18) |
| EORTC recurrence | |
| 1-4 | 35 (87.5) |
| 5-9 | 5 (12.5) |
| Median (min, max) | 3 (1-7) |

TABLE 3

| | FAS | |
|---|---|---|
| Variable | n (%) | |
| Sex | | |
| Female | 4 | (10.0) |
| Male | 36 | (90.0) |
| WHO performance score | | |
| 0 | 35 | (87.5) |
| 1 | 5 | (12.5) |
| Smoking status | | |
| Current smoker | 10 | (25.0) |
| Former smoker | 11 | (27.5) |
| Non-smoker | 18 | (45.0) |
| Not available | 1 | (2.5) |
| BCG maintenance performed during previous therapy | | |
| No | 26 | (65.0) |
| Yes | 14 | (35.0) |
| T classification for recurrence | | |
| T1 | 7 | (17.5) |
| T1/CIS | 5 | (12.5) |
| Ta | 6 | (15.0) |
| Ta/CIS | 5 | (12.5) |
| CIS | 17 | (42.5) |
| 1973 WHO grading of recurrence | | |
| Grade 2 | 3 | (7.5) |
| Grade 3 | 37 | (92.5) |
| 2004 WHO grading of recurrence for study inclusion | | |
| High-grade urothelial carcinoma | 40 | (100.0) |
| EORTC progression score | | |
| 7-13 | 12 | (30.0) |
| 14-23 | 28 | (70.0) |
| Median (min, max) | 16 | (7, 20) |
| EORTC recurrence score | | |
| 1-4 | 4 | (10.0) |
| 5-9 | 30 | (75.0) |
| 10-17 | 6 | (15.0) |
| Median (min, max) | 8 | (4, 11) |
| Baseline PPD test | | |
| No reaction | 31 | (77.5%) |
| Positive | 7 | (17.5%) |
| Missing | 2 | (5.0%) |

TABLE 4

| | no | yes/former | |
|---|---|---|---|
| Smoking | (n = 18) | (n = 21) | p |
| Recurrence-free rate in | 53.1 | 45.5 | 0.652 |
| the bladder at 60 weeks | [27.8, 73.2] | [22.4, 66.1] | |
| in % [95% CI] | | | |
| | no | yes | |
| CIS | (n = 13) | (n = 27) | p |
| Recurrence-free rate in | 61.5 | 42.4 | 0.289 |
| the bladder at 60 weeks | [30.8, 81.8] | [22.0, 61.5] | |
| in % [95% CI] | | | |
| Previous BCG | no | yes | |
| maintenance | (n = 26) | (n = 14) | p |
| Recurrence-free rate in | 54.3 | 38.4 | 0.371 |
| the bladder at 60 weeks | [32.9, 71.6] | [12.2, 64.6] | |
| in % [95% CI] | | | |
| BCG unresponsive | no | yes | |
| (FDA definition) | (n = 24) | (n = 16) | p |
| Recurrence-free rate in | 63.6% | 24.0% | 0.022 |
| the bladder at 60 weeks | [40.3%, 79.9%] | [5.9%, 48.8%] | |
| in % [95% CI] | | | |
| | no | PPD− to PPD+ | |
| PPD conversion | (n = 15) | (n = 5) | p |
| Recurrence-free rate in | 62.9% | 80.0% | 0.505 |
| the bladder at 60 weeks | [32.3%, 82.6%] | [20.4%, 96.9%] | |
| in % [95% CI] | | | |

TABLE 5

| Treatment | n (%) | |
|---|---|---|
| Cystectomy | 13* | (32.5) |
| BCG (upper tract) | 2 | (5.0) |
| Systemic chemotherapy | 3 | (7.5) |
| Intravesical chemotherapy | 6* | (15.0) |

TABLE 6

| | Grade 1 | Grade 2 | Grade 3 |
|---|---|---|---|
| AE | n (%) | n (%) | n (%) |
| Vertigo | | 1 (2.4) | |
| Gastrointestinal disorders | 2 (4.8) | | |
| Fatigue | 2 (4.8) | 3 (7.1) | |
| Fever | 2 (4.8) | 1 (2.4) | |
| Frequency, urgency | 7 (16.7) | 5 (11.9) | |
| Malaise | | 1 (2.4) | |
| BCG induced systemic inflammatory | | 1 (2.4) | |
| reaction | | | |
| Cold | | 1 (2.4) | |
| GU infection | | 14 (33.3) | 2 (4.8) |
| Alanine aminotransferase increased | | 1 (2.4) | |
| Neuralgia | | 1 (2.4) | |
| Hematuria | 2 (4.8) | | |
| Macrohematuria | 1 (2.4) | | |
| Urinary retention | 1 (2.4) | | |
| Urinary tract obstruction | 1 (2.4) | 1 (2.4) | |
| Urinary tract pain | 6 (14.3) | 1 (2.4) | |
| Vaginal pain | | 1 (2.4) | |
| Skin affection | 3 (7.1) | 2 (4.8) | |
| Thromboembolic event | | 1 (2.4) | |

The sequences listed as SEQ ID NO.1 and SEQ ID NO.2 are as follows:

SEQ ID NO. 1:
Nucleotide sequence encoding a phagolysosomal domain (nt 211-1722) and
a stop codon (nt 1879-1881)
atgacagacg tgagccgaaa gattcgagct tggggacgcc gattgatgat cggcacggca
gcggctgtag tccttccggg cctggtgggg cttgccggcg gagcggcaac cgcgggcgcg
ttctcccggc cggggctgcc ggtcgagtac ctgcagtctg caaagcaatc cgctgcaaat
aaattgcact cagcaggaca aagcacgaaa gatgcatctg cattcaataa agaaaattca
atttcatcca tggcaccacc agcatctccg cctgcaagtc ctaagacgcc aatcgaaaag
aaacacgcgg atgaaatcga taagtatata caaggattgg attacaataa aaacaatgta
ttagtatacc acggagatgc agtgacaaat gtgccgccaa gaaaaggtta caaagatgga
aatgaatata ttgttgtgga gaaaaagaag aaatccatca atcaaaataa tgcagacatt
caagttgtga atgcaatttc gagcctaacc tatccaggtg ctctcgtaaa agcgaattcg
gaattagtag aaaatcaacc agatgttctc cctgtaaaac gtgattcatt aacactcagc
attgatttgc caggtatgac taatcaagac aataaaatcg ttgtaaaaaa tgccactaaa
tcaaacgtta acaacgcagt aaatacatta gtggaaagat ggaatgaaaa atatgctcaa
gcttatccaa atgtaagtgc aaaaattgat tatgatgacg aaatggctta cagtgaatca
caattaattg cgaaatttgg tacagcattt aaagctgtaa ataatagctt gaatgtaaac
ttcggcgcaa tcagtgaagg gaaaatgcaa gaagaagtca ttagttttaa acaaatttac
tataacgtga atgttaatga acctacaaga ccttccagat ttttcggcaa agctgttact
aaagagcagt tgcaagcgct tggagtgaat gcagaaaatc ctcctgcata tatctcaagt
gtggcgtatg gccgtcaagt ttatttgaaa ttatcaacta attcccatag tactaaagta
aaagctgctt ttgatgctgc cgtaagcgga aaatctgtct caggtgatgt agaactaaca
aatatcatca aaaattcttc cttcaaagcc gtaatttacg gaggttccgc aaaagatgaa
gttcaaatca tcgacggcaa cctcggagac ttacgcgata ttttgaaaaa aggcgctact
tttaatcgag aaacaccagg agttcccatt gottatacaa caaacttcct aaaagacaat
gaattagctg ttattaaaaa caactcagaa tatattgaaa caacttcaaa agcttataca
gatggaaaaa ttaacatcga tcactctgga ggatacgttg ctcaattcaa catttcttgg
gatgaagtaa attatgatcc tgaaggtaac gaaattgttc aacataaaaa ctggagcgaa
aacaataaaa gcaagctagc tcatttcaca tcgtccatct atttgccagg taacgcgaga
aatattaatg tttacgctaa agaatgcact ggtttagctt gggaatggtg gagaacggta
attgatgacc ggaacttacc acttgtgaaa aatagaaata tctccatctg gggcaccacg
ctttatccga aatatagtaa taaagtagat aatccaatcg aatatgcatt agcctatgga
agtcagggtg atcttaatcc attaattaat gaaatcagca aaatcatttc agctgcagtt
ctttcctctt taacatcgaa gctacctgca gagttcgtta ggcgcggatc cggaattcga
agcttatcga tgtcgacgta g
SEQ ID NO. 2:
Amino acid sequence encoded by the nucleotide sequence of SEQ ID NO: 1
Met Thr Asp Val Ser Arg Lys Ile Arg Ala Trp Gly Arg Arg Leu Met Ile Gly Thr
Ala Ala Ala Val Val Leu Pro Gly Leu Val Gly Leu Ala Gly Gly Ala Ala Thr Ala
Gly Ala Phe Ser Arg Pro Gly Leu Pro Val Glu Tyr Leu Gln Ser Ala Lys Gln
Ser Ala Ala Asn Lys Leu His Ser Ala Gly Gln Ser Thr Lys Asp Ala Ser Ala
Phe Asn Lys Glu Asn Ser Ile Ser Ser Met Ala Pro Pro Ala Ser Pro Pro Ala
Ser Pro Lys Thr Pro Ile Glu Lys Lys His Ala Asp Glu Ile Asp Lys Tyr Ile Gln
Gly Leu Asp Tyr Asn Lys Asn Asn Val Leu Val Tyr His Gly Asp Ala Val Thr
Asn Val Pro Pro Arg Lys Gly Tyr Lys Asp Gly Asn Glu Tyr Ile Val Val Glu Lys
Lys Lys Lys Ser Ile Asn Gln Asn Asn Ala Asp Ile Gln Val Val Asn Ala Ile Ser
Ser Leu Thr Tyr Pro Gly Ala Leu Val Lys Ala Asn Ser Glu Leu Val Glu Asn
Gln Pro Asp Val Leu Pro Val Lys Arg Asp Ser Leu Thr Leu Ser Ile Asp Leu
Pro Gly Met Thr Asn Gln Asp Asn Lys Ile Val Val Lys Asn Ala Thr Lys Ser
Asn Val Asn Asn Ala Val Asn Thr Leu Val Glu Arg Trp Asn Glu Lys Tyr Ala
Gln Ala Tyr Pro Asn Val Ser Ala Lys Ile Asp Tyr Asp Asp Glu Met Ala Tyr Ser
Glu Ser Gln Leu Ile Ala Lys Phe Gly Thr Ala Phe Lys Ala Val Asn Asn Ser
Leu Asn Val Asn Phe Gly Ala Ile Ser Glu Gly Lys Met Gln Glu Glu Val Ile Ser
Phe Lys Gln Ile Tyr Tyr Asn Val Asn Val Asn Glu Pro Thr Arg Pro Ser Arg
Phe Phe Gly Lys Ala Val Thr Lys Glu GIn Leu Gln Ala Leu Gly Val Asn Ala
Glu Asn Pro Pro Ala Tyr Ile Ser Ser Val Ala Tyr Gly Arg Gln Val Tyr Leu Lys
Leu Ser Thr Asn Ser His Ser Thr Lys Val Lys Ala Ala Phe Asp Ala Ala Val
Ser Gly Lys Ser Val Ser Gly Asp Val Glu Leu Thr Asn Ile Ile Lys Asn Ser Ser
Phe Lys Ala Val Ile Tyr Gly Gly Ser Ala Lys Asp Glu Val Gln Ile Ile Asp Gly
Asn Leu Gly Asp Leu Arg Asp Ile Leu Lys Lys Gly Ala Thr Phe Asn Arg Glu
Thr Pro Gly Val Pro Ile Ala Tyr Thr Thr Asn Phe Leu Lys Asp Asn Glu Leu
Ala Val Ile Lys Asn Asn Ser Glu Tyr Ile Glu Thr Thr Ser Lys Ala Tyr Thr Asp
Gly Lys Ile Asn Ile Asp His Ser Gly Gly Tyr Val Ala GIn Phe Asn Ile Ser Trp
Asp Glu Val Asn Tyr Asp Pro Glu Gly Asn Glu Ile Val Gln His Lys Asn Trp
Ser Glu Asn Asn Lys Ser Lys Leu Ala His Phe Thr Ser Ser Ile Tyr Leu Pro
Gly Asn Ala Arg Asn Ile Asn Val Tyr Ala Lys Glu Cys Thr Gly Leu Ala Trp Glu
Trp Trp Arg Thr Val Ile Asp Asp Arg Asn Leu Pro Leu Val Lys Asn Arg Asn Ile
Ser Ile Trp Gly Thr Thr Leu Tyr Pro Lys Tyr Ser Asn Lys Val Asp Asn Pro Ile
Glu Tyr Ala Leu Ala Tyr Gly Ser Gln Gly Asp Leu Asn Pro Leu Ile Asn Glu Ile
Ser Lys Ile Ile Ser Ala Ala Val Leu Ser Ser Leu Thr Ser Lys Leu Pro Ala Glu
Phe Val Arg Arg Gly Ser Gly Ile Arg Ser Leu Ser Met Ser Thr

1. A recombinant Mycobacterium cell, which is a urease-deficient, recombinant M. bovis BCG cell from strain Danish subtype Prague, which comprises a recombinant nucleic acid molecule encoding a fusion polypeptide comprising:
(a) a domain capable of eliciting an immune response, and
(b) a phagolysosomal escape domain
in combination with a carrier suitable for administration as an immunotherapeutic agent to a subject suffering from bladder carcinoma, as a second-line therapy.

2. The recombinant Mycobacterium cell of claim 1 wherein said recombinant Mycobacterium cell is a urease-deficient, recombinant M. bovis BCG cell from strain Danish subtype Prague, which comprises a recombinant nucleic acid molecule encoding a fusion polypeptide comprising:
(a) a domain capable of eliciting an immune response comprising the amino acid sequence from aa.41 to aa.51 in SEQ ID No.2, and
(b) a Listeria phagolysosomal escape domain encoded by a nucleic acid molecule selected from
(i) a nucleotide sequence comprising nucleotides 211-1722 as shown in SEQ ID No.1,
(ii) a nucleotide sequence which encodes the same amino acid sequence as the sequence from (i), and
(iii) a nucleotide sequence hybridizing under stringent conditions with the sequence from (i) or (ii).

3. The recombinant Mycobacterium cell of claim 1, wherein the recombinant nucleic acid molecule does not comprise any functional selection marker.

4-20. (canceled)

21. A method for the immunotherapeutic treatment of bladder carcinoma, particularly of non-muscle-invasive bladder carcinoma in a subject in need thereof, comprising administering to said subject as a second-line therapy a recombinant Mycobacterium cell, which is a urease-deficient, recombinant M. bovis BCG cell, which comprises a recombinant nucleic acid molecule encoding a fusion polypeptide comprising:
(a) a domain capable of eliciting an immune response, and
(b) a Listeria phagolysosomal escape domain.

22. A method for the immunotherapeutic treatment of bladder carcinoma, in a human subject in need thereof, comprising administering to said subject as a second-line therapy a recombinant Mycobacterium cell, which is a urease-deficient, recombinant M. bovis BCG cell from strain Danish subtype Prague, which comprises a recombinant nucleic acid molecule encoding a fusion polypeptide comprising:
(a) a domain capable of eliciting an immune response comprising the amino acid sequence from aa.41 to aa.51 in SEQ ID No.2, and
(b) a Listeria phagolysosomal escape domain encoded by a nucleic acid molecule selected from
(i) a nucleotide sequence comprising nucleotides 211-1722 as shown in SEQ ID No.1,
(ii) a nucleotide sequence which encodes the same amino acid sequence as the sequence from (i), and
(iii) a nucleotide sequence hybridizing under stringent conditions with the sequence from (i) or (ii).

23. The method of claim 22, wherein the human subject is suffering from non-muscle-invasive bladder carcinoma (NMIBC).

24. The method of claim 22, wherein the human subject is suffering from recurrent bladder carcinoma.

25. The method of claim 22, wherein the human subject has relapsed and/or has progressed after a first treatment of bladder carcinoma.

26. The method of claim 22, wherein the administration comprises vesicular instillation into said human subject's urinary bladder.

27. The method of claim 22, wherein the human subject has previously undergone at least one unsuccessful first-line therapy of bladder carcinoma.

28. The method of claim 22, wherein said human subject has previously undergone at least one unsuccessful first-line therapy of bladder carcinoma comprising an immunotherapy.

29. The method of claim 28, wherein said at least one unsuccessful first-line therapy of bladder carcinoma comprising an immunotherapy is an immunotherapy with standard BCG.

30. The method of claim 22, wherein said human subject has previously undergone at least one unsuccessful first-line therapy of bladder carcinoma comprising cystectomy or another local treatment and/or systemic chemotherapy.

31. The method of claim 22, wherein said human subject has previously undergone at least one unsuccessful first-line therapy of bladder carcinoma selected from the group consisting of cisplatin-based chemotherapy, cisplatin-based chemotherapy followed by bladder surgery and/or radiation therapy, concomitant chemotherapy and standard BCG.

32. The method of claim 22, wherein the subject is a smoker.

33. The method of claim 22, wherein the immunotherapeutic agent is administered into the bladder according to a schedule involving weekly instillations

34. The method of claim 22, wherein the immunotherapeutic agent is administered into the bladder according to a schedule involving weekly instillations during (i) an induction phase and (ii) a maintenance phase of at least one year.

35. The method of claim 34, wherein (i) the induction phase has 6 weekly instillations, (ii) the maintenance phase of at least one year has a first maintenance phase after about 3 months with 3 weekly instillations, a second maintenance phase after about 6 months with 3 weekly instillations and a third maintenance phase after about 12 months with 3 weekly instillations.

36. The method of claim 22, wherein the recombinant Mycobacterium cell is administered at a dose of from about 10⁷ to 5×10⁹ CFU.

37. The method of claim 22, wherein the recombinant Mycobacterium cell is administered at a dose of from about 2×10⁹ CFU per administration.

38. The method of claim 22, wherein disease recurrence is inhibited for a time period selected from the group consisting of at least 1 year, at least 2 years, at least 3 years and at least 4 years.

39. The method of claim 22, wherein the immunotherapy provides a disease-free time period selected from the group consisting of at least 1 year, at least 2 years, at least 3 years and at least 4 years, for at least 30% of the patients.

40. The method of claim 39, wherein the immunotherapy provides a disease-free time period selected from the group consisting of at least 1 year, at least 2 years, at least 3 years and at least 4 years, for at least 45% of the patients.

41. The method of claim 22, wherein disease recurrence is blocked for at least one further year in a subject without disease recurrence for a time period of 2 years after treatment starts.

42. The method of claim 22, wherein overall survival is increased compared to a human subject without treatment in a time period of at least 1 year.

43. The method of claim 42, wherein overall survival is increased compared to a human subject without treatment in a time period of at least 2 years, of at least years or of at least 4 years.

44. The method of claim 22, wherein the need of cystectomy is reduced or avoided for a time period selected from the group consisting of at least a 1 year, at least 2 years, at least 3 years and at least 4 years; in at least 30% of the patients.

45. The method of claim 44, wherein the need of cystectomy is reduced or avoided for at least 45% of the patients.
